(19) United States
(12) Patent Application Publication
(10) Pub. No.: US 2023/0162864 A1
(43) Pub. Date: May 25, 2023

(54) EXPLORATION TOOL FOR PREDICTING THE IMPACT OF RISK FACTORS ON HEALTH OUTCOMES

(71) Applicant: KONINKLIJKE PHILIPS N.V., EINDHOVEN (NL)

(72) Inventors: Eran SIMHON, Boston, MA (US); Reza SHARIFI SEDEH, Malden, MA (US)

(21) Appl. No.: 17/422,790

(22) PCT Filed: Jan. 14, 2020
(86) PCT No.: PCT/EP2020/050783

§ 371 Date: Jul. 14, 2021

Related U.S. Application Data
(60) Provisional application No. 62/792,454, filed on Jan. 15, 2019.

(51) Int. Cl. G16H 50/30; G06F 17/18

(52) U.S. Cl. CPC G16H 50/30; G06F 17/18; G16H 10/20

(57) ABSTRACT

A method for identifying risk factors that have an impact on health outcomes, including: receiving, by a graphical user interface (GUI), from a user features of similarity, a risk factor, and a key performance indicator (KPI); receiving, by the GUI, from the user values for the features of similarity and risk factor; selecting, by a processor, patient data including features of similarity data, risk factor data, and KPI data; and determining, by the processor, the optimal features of similarity by optimizing the minimum value of an average standard deviation (STD) of the KPI based upon the received user features of similarity, received risk factor, and the received values for the features of similarity and risk factor.

### TECHNICAL FIELD

Various exemplary embodiments disclosed herein relate generally to an exploration tool for predicting the impact of risk factors on health outcomes.

### BACKGROUND

Identifying risk factors associated with the health outcomes of patients has been a challenge for many years. The rise of precision medicine further pushes the need for more accurate risk prediction that focuses on a patient's specific medical situation. One of the approaches to determine a risk score for a patient is to compare retrospectively health outcomes of patients that have the specific risk factor and patients that do not a have the specific risk factor. Then, another challenge arises: in order to have a valid comparison, patients that are compared to each other should be in some degree similar to each other and they should differ only by one specific risk factor. For that, several techniques have been proposed such as propensity score matching and patient similarity networks. These techniques are complex and are not easily interpretable by health care personal. The approach disclosed below is based on a statistical comparison that can be easily understood by patients and clinicians.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a method for identifying risk factors that have an impact on health outcomes, including: receiving, by a graphical user interface (GUI), from a user features of similarity, a risk factor, and a key performance indicator (KPI); receiving, by the GUI, from the user values for the features of similarity and risk factor; selecting, by a processor, patient data including features of similarity data, risk factor data, and KPI data; and determining, by the processor, the optimal features of similarity by optimizing the minimum value of an average standard deviation (STD) of the KPI based upon the received user features of similarity, received risk factor, and the received values for the features of similarity and risk factor.

Various embodiments are described, wherein the user features of similarity include mandatory features and optional features.

Various embodiments are described, wherein average STD of the KPI is calculated as

$$average STD = \frac{\sqrt{{STD}_{1}^{2} + {STD}_{2}^{2} + … + {STD}_{n}^{2} …}}{n}$$

where STDₙ is the standard deviation of the KPI for each group of patients, where each group of patients are in a same KPI group.

Various embodiments are described, wherein optimizing the minimum value of an average STD of the KPI includes using a genetic algorithm.

Various embodiments are described, further including computing KPI differences.

Various embodiments are described, wherein the KPI differences include one of a 95% confidence interval and a two-sided t-test.

Various embodiments are described, further including presenting the KPI differences to the user.

Various embodiments are described, further including receiving user input to modify the features of similarity and then determining the optimal features of similarity by optimizing the minimum value of an average standard deviation (STD) of the KPI based upon the modified features of similarity.

Further various embodiments relate to a non-transitory machine-readable storage medium encoded with instructions for identifying risk factors that have an impact on health outcomes, including: instructions for receiving, by a graphical user interface (GUI), from a user features of similarity, a risk factor, and a key performance indicator (KPI); instructions for receiving, by the GUI, from the user values for the features of similarity and risk factor; instructions for selecting, by a processor, patient data including features of similarity data, risk factor data, and KPI data; and

instructions for determining, by the processor, the optimal features of similarity by optimizing the minimum value of an average standard deviation (STD) of the KPI based upon the received user features of similarity, received risk factor, and the received values for the features of similarity and risk factor.

Various embodiments are described, wherein the user features of similarity include mandatory features and optional features.

Various embodiments are described, wherein average STD of the KPI is calculated as

$$average STD = \frac{\sqrt{{STD}_{1}^{2} + {STD}_{2}^{2} + … + {STD}_{n}^{2} …}}{n}$$

where STDₙ is the standard deviation of the KPI for each group of patients, where each group of patients are in a same KPI group.

Various embodiments are described, wherein instructions for optimizing the minimum value of an average STD of the KPI includes using a genetic algorithm.

Various embodiments are described, further including instructions for computing KPI differences.

Various embodiments are described, wherein the KPI differences include one of a 95% confidence interval and a two-sided t-test.

Various embodiments are described, further including instructions for presenting the KPI differences to the user.

Various embodiments are described, further including instructions for receiving user input to modify the features of similarity and then determining the optimal features of similarity by optimizing the minimum value of an average standard deviation (STD) of the KPI based upon the modified features of similarity.

### BRIEF DESCRIPTION OF THE DRAWINGS

In order to better understand various exemplary embodiments, reference is made to the accompanying drawings, wherein:

FIG. 1 illustrates a flow diagram of the operation of exploration tool;

FIG. 2 illustrates a graphical user interface (GUI) that may be used by the user to select feature(s) used for similarity, a risk factor to explore, and a key performance indicator (KPI) to use; and

FIG. 3 illustrates a user interface for a user to input values for the selected mandatory and optional features.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

Embodiments of an exploration tool are described herein that implements a technique to find sets of similar patients so the impact of a specific factor (e.g., clinical, behavioral, demographic, etc.) may be learned at a more accurate level. For example, it is known for many years that smoking negatively affects many health outcomes: however, to understand the impact on patients with different clinical and behavioral factors requires clustering patients into similarity groups. While other clustering techniques use unsupervised learning (i.e., they are not minimizing a loss function), the approach implemented by the exploration tool described herein has the physician/care giver select a specific health outcome and cluster patients in a way that minimizes the standard deviation of that health outcome. Furthermore, an advantage of exploration tool, is in the interpretability of the results presented to the user. The results are not based on machine learning or network analysis but purely on statistical comparison. Thus, the physician/care giver may easily understand and trust the results.

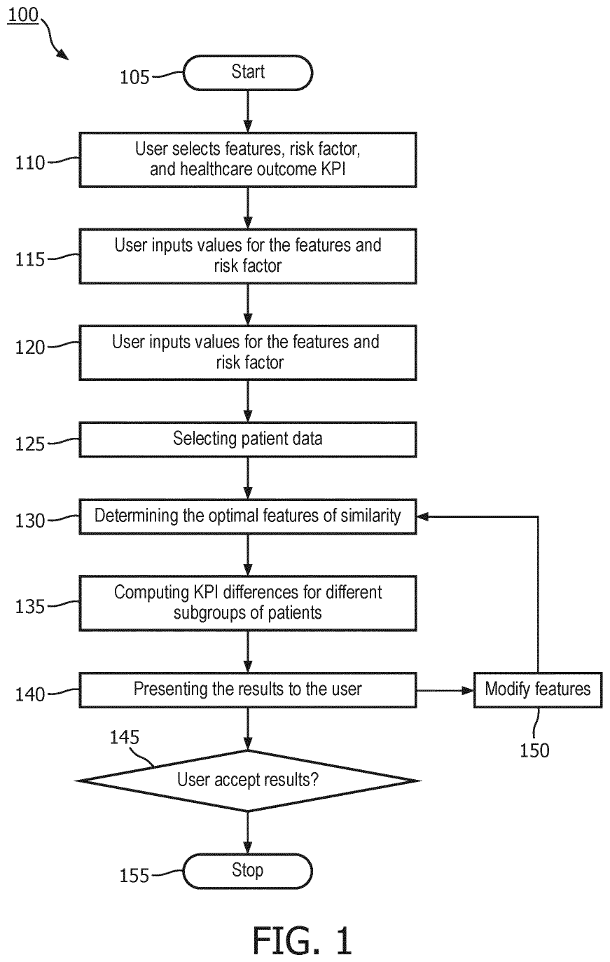

FIG. 1 illustrates a flow diagram of the operation of exploration tool 100. The operation begins at 105, and first, the user selects 110, features of similarity, a specific risk factor to explore, and a specific health outcome KPI, where the goal is to understand the impact of the selected risk factor on the selected health outcome KPI. Then the user inputs values for the features of similarity and the risk factor 115. Next, the exploration tool 100 selects patient data 125 based upon the user inputs. Then, an algorithm (described in detail below) finds an optimal set of features that results in an optimal set of patients that are similar to the given patients but includes patients with and without the selected factor(s) 130. Then the exploration tool computes KPI differences for the different subgroups of patients. Next, a user interface visualizes for the user how the selected factor(s) impacts patients that are similar to the given patient by presenting the results to the user 140. For example, the solution may be used to learn how smoking impacts health care cost of congestive heart failure (CHF) patients with high cholesterol. Finally, if the user accepts the results 145, the operation ends at 155. Otherwise, the user modifies the feature(s) selected 150 and then then exploration tool again determines the optimal features of similarity 130. These steps will now be described in greater detail.

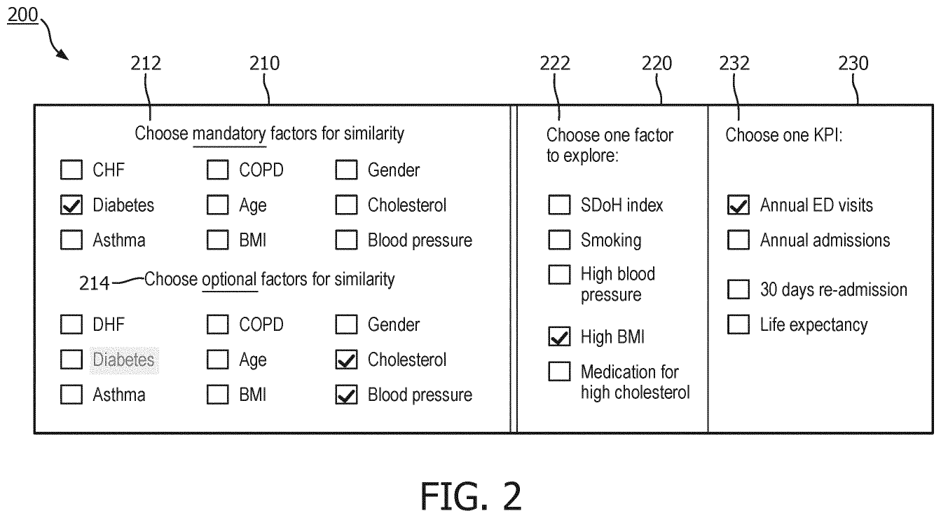

FIG. 2 illustrates a graphical user interface (GUI) that may be used by the user to select feature(s) used for similarity, a risk factor to explore, and a key performance indicator (KPI) to use. The GUI 200 may include a feature pane 210, a risk factor pane 220, and a KPI pane 230. The user may select a set of mandatory features of similarity 212 using the risk factor pane 210. In this example, the mandatory features may include congestive heart failure (CHF), diabetes, asthma, chronic obstructive pulmonary disease (COPD), age, body mass index (BMI), gender, cholesterol, and blood pressure, but other features may be listed as well. The similarity group will include only patients that have these selected features. The user may also select a set of optional features of similarity 214 using the features pane 210. The same list of features, as is shown in FIG. 2, may be listed or a different set of features may instead be listed. Note that any features selected as a mandatory feature will not be available to be selected as an optional feature. This is shown in FIG. 2 where diabetes is greyed out and cannot be selected as an optional feature for similarity 214. The algorithm described below will determine which of these features will be used for creating similarity groups. The user is also able to select one risk factor 222 to explore using the risk factor pane 220. This risk factor may be clinical, operational, or social-behavioral. In FIG. 2, the risk factors to explore include social determinants of health (SDoH), smoking, high blood pressure, high BMI, and medication for high cholesterol, but other risk factors may be listed as well. Finally, the user may select one health outcome as indicated by a KPI 232 using the KPI pane 230. The health outcome may be clinical or operational. In FIG. 2, the KPIs include annual emergency department (ED) visits, annual admissions, 30-day re-admission, and life expectancy, but other examples of KPIs may be used as well.

For example, assume that a physician is interested to learn about the impact of high BMI on ED utilization of patients with diabetes, high cholesterol, and high blood pressure. However, the physician does not know a priori whether the latter two factors have any impact on ED utilization of diabetes patients. In this case, the physician will select high BMI as the risk factor to explore; ED utilization as the health outcome; diabetes as a mandatory feature; and high cholesterol and high blood pressure as optional features. The selection of these items is shown by the checkmarks in FIG. 2.

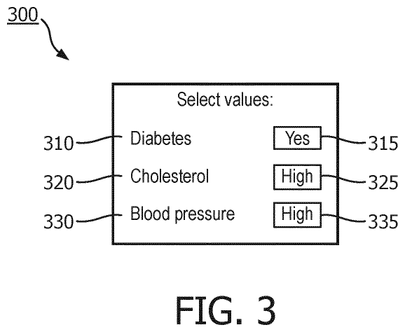

After the user selects the mandatory and optional features, they are prompted to select a value for each feature. FIG. 3 illustrates a user interface for a user to input values for the selected mandatory and optional features. In FIG. 3, the features diabetes 310, cholesterol 320, and blood pressure 330 are shown corresponding to the features selected in FIG. 2. The user inputs values for each of these features 315, 325, and 335 respectively.

Next, the exploration tool aggregates data from patient electronic medical record (EMR) data stored in an EMR database. From the EMR data, a dataset is created where each row represents one patient and each column represents one feature. The dataset will include the selected risk factor, the selected health outcome, and the selected mandatory and optional features. Table 1 below illustrates an example of the output of this step based on the example provided in FIGS. 2 and 3. Table 1 has a first column with a patient ID. Then there are columns for BMI, diabetes, cholesterol, blood pressure, and the number of ED visits per year with the data value for each corresponding patient.

TABLE 1

| Patient | | | | Blood | ED visits |
|---|---|---|---|---|---|
| ID | BMI | Diabetes | Cholesterol | Pressure | per year |
| 1 | High | Yes | High | High | 2 |
| 2 | High | No | Normal | High | 0 |
| 3 | Normal | Yes | Normal | High | 1 |
| 4 | Low | Yes | High | normal | 3 |
| 5 | High | Yes | High | High | 3 |
| 6 | Low | Yes | High | normal | 4 |
| 7 | Low | Yes | High | High | 4 |
| . . . | . . . | . . . | . . . | . . . | . . . |

Then the exploration tool computes a standard deviation. Given the input from the user about which factors to use, a binary vector X is created that determines which factors should be considered for similarity (1) and which should not (0). For example, if X={1,1,0} then the factors diabetes and cholesterol should be considered, and blood pressure should be ignored. This convention follows the order of the columns shown in Table 1. A vector α is also created that contains the value of each feature as defined by the user using GUIs, when continuing to use the example from the first row of Table 1, α={yes, high, high}. Given α and X, the standard deviation (STD) of the selected health outcome of all patients split to subsets is calculated, where each subset has different value of the selected factor. For example, if the factor is BMI, there will be three subsets: high BMI, normal BMI and low BMI. Given that X={1,0,1} and α={yes, high, high}, the standard deviation (STD) of ED visits will be calculated for three KPI groups: 1) all patients with Diabetes, high blood pressure and low BMI; 2) all patients with Diabetes, high blood pressure and normal BMI; and 3) all patients with Diabetes, high blood pressure and high BMI. Because there are multiple STD values (one for each subset), the Euclidian norm is used to calculate an average STD. By using the Euclidian distance, higher STDs get higher weight when computing the average STD Assume there are n factors, then the average STD is calculated as:

$$average STD = \frac{\sqrt{{STD}_{1}^{2} + {STD}_{2}^{2} + … + {STD}_{n}^{2} …}}{n} .$$

The exploration tool next optimizes the standard deviation. The goal of this step is to identify which features should be included for similarity and which should be ignored, i.e., finding the optimal value of X. Choosing very few features will lead to a group that is too general with larger variation. On the other hand, choosing many features will lead to a group that is too narrow and maybe inaccurate comparison. To overcome this issue the set of features that minimizes the average STD is found. Therefore, a feature will be included if it leads to a more homogenous group with regard to the selected health outcome. The genetic algorithm may be used for this step where the average STD is the objective function and the elements of the vector X are the control variables. A vector γ is defined that determines which of the features are optional. Continuing the previous example from Table 1, γ={2,3} since the first feature (diabetes) was defined as mandatory, by the physician, while the other two were defined as optional. In this case, the feasible set of X is {1,1,1}, {1,1,0}, {1,0,1} or {1,0,0}. In other scenarios, the number of features used may be much larger, leading to a large number of combination of features. The optimization problem minimizes the average STD subjection to constraints as follows:
min(average STD)
subject to βᵢ∈{0,1} ∀i=γ,
   where βᵢ is are the optional bit positions in X.

To find the optimal solution, the genetic algorithm is used, because this algorithm may work with any form of objective function and with binary variables. The output of this optimization step is the optimal value of X, denoted by Xₒₚₜ.

Next, Xₒₚₜ may be used to compute KPI differences. At this step, Xₒₚₜ may be used to compute the average health outcome for each subset and the 95% confidence interval. Also, a two-sided t-test may be performed to see if the difference between the different KPI values of each subset are significant. For example, if Xₒₚₜ={1,0,1}, the 95% confidence interval and the two-sided t-test would be as shown in Tables 2 and 3 below.

TABLE 2
Average number of ED visits per year (95% CI)
patient with Diabetes and high blood pressure

| Low BMI | Normal BMI | High BMI |
|---|---|---|
| (625 patients) | (1468 patients) | (2078 patients) |
| 0.861 ± 0.088 | 0.631 ± 0.047 | 0.95 ± 0.031 |

TABLE 3

| | Low BMI | Normal BMI |
|---|---|---|
| Normal BMI | P-value = 0.004 | — |
| High BMI | P-value = 0.003 | P-value = 0.00002 |

From this result, the user can conclude that BMI had significant impact on patient with diabetes and high blood pressure, while splitting patients also by their cholesterol level does not create more homogenous group.

The exploration tool may be used on the population level to identify patient groups that are in highest risk. The exploration tool may also be used on the patient level to create a more accurate risk estimation based on the patient clinical/behavioral factors by comparing the patient to similar patients.

The exploration tool solves the technological problem of identifying the impact of risk factors on health outcomes. Often such identification has been done using machine learning, but the exploration tools uses patient clustering to minimize variation to identify the impact of risk factors on health outcome. Such an approach provides results that are more easily understood by users of the exploration tool.

The embodiments described herein may be implemented as software running on a processor with an associated memory and storage. The processor may be any hardware device capable of executing instructions stored in memory or storage or otherwise processing data. As such, the processor may include a microprocessor, field programmable gate array (FPGA), application-specific integrated circuit (ASIC), graphics processing units (GPU), specialized neural network processors, cloud computing systems, or other similar devices.

The memory may include various memories such as, for example L1, L2, or L3 cache or system memory. As such, the memory may include static random-access memory (SRAM), dynamic RAM (DRAM), flash memory, read only memory (ROM), or other similar memory devices.

The storage may include one or more machine-readable storage media such as read-only memory (ROM), random-access memory (RAM), magnetic disk storage media, optical storage media, flash-memory devices, or similar storage media. In various embodiments, the storage may store instructions for execution by the processor or data upon with the processor may operate. This software may implement the various embodiments described above.

Further such embodiments may be implemented on multiprocessor computer systems, distributed computer systems, and cloud computing systems. For example, the embodiments may be implemented as software on a server, a specific computer, on a cloud computing, or other computing platform.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A method for identifying risk factors that have an impact on health outcomes, comprising:
receiving, by a graphical user interface (GUI), from a user features of similarity, a risk factor, and a key performance indicator (KPI);
receiving, by the GUI, from the user values for the features of similarity and risk factor;
selecting, by a processor, patient data including features of similarity data, risk factor data, and KPI data; and
determining, by the processor, the optimal features of similarity by optimizing the minimum value of an average standard deviation (STD) of the KPI based upon the received user features of similarity, received risk factor, and the received values for the features of similarity and risk factor.

2. The method of claim 1, wherein the user features of similarity include mandatory features and optional features.

3. The method of claim 1, wherein average STD of the KPI is calculated as
$$average STD = \frac{\sqrt{{STD}_{1}^{2} + {STD}_{2}^{2} + … + {STD}_{n}^{2} …}}{n}$$
where STDₙ is the standard deviation of the KPI for each group of patients, where each group of patients are in a same KPI group.

4. The method of claim 1, wherein optimizing the minimum value of an average STD of the KPI includes using a genetic algorithm.

5. The method of claim 1, further comprising computing KPI differences.

6. The method of claim 5, wherein the KPI differences include one of a 95% confidence interval and a two-sided t-test.

7. The method of claim 5, further comprising presenting the KPI differences to the user.

8. The method of claim 7, further comprising receiving user input to modify the features of similarity and then determining the optimal features of similarity by optimizing the minimum value of an average standard deviation (STD) of the KPI based upon the modified features of similarity.

9. A non-transitory machine-readable storage medium encoded with instructions for identifying risk factors that have an impact on health outcomes, comprising:
instructions for receiving, by a graphical user interface (GUI), from a user features of similarity, a risk factor, and a key performance indicator (KPI);
instructions for receiving, by the GUI, from the user values for the features of similarity and risk factor;
instructions for selecting, by a processor, patient data including features of similarity data, risk factor data, and KPI data; and
instructions for determining, by the processor, the optimal features of similarity by optimizing the minimum value of an average standard deviation (STD) of the KPI based upon the received user features of similarity, received risk factor, and the received values for the features of similarity and risk factor.

10. The non-transitory machine-readable storage medium of claim 9, wherein the user features of similarity include mandatory features and optional features.

11. The non-transitory machine-readable storage medium of claim 9, wherein average STD of the KPI is calculated as
$$average STD = \frac{\sqrt{{STD}_{1}^{2} + {STD}_{2}^{2} + … + {STD}_{n}^{2} …}}{n}$$
where STDₙ is the standard deviation of the KPI for each group of patients, where each group of patients are in a same KPI group.

12. The non-transitory machine-readable storage medium of claim 9, wherein instructions for optimizing the minimum value of an average STD of the KPI includes using a genetic algorithm.

13. The non-transitory machine-readable storage medium of claim 1, further comprising instructions for computing KPI differences.

14. The non-transitory machine-readable storage medium of claim 5, wherein the KPI differences include one of a 95% confidence interval and a two-sided t-test.

15. The non-transitory machine-readable storage medium of claim 5, further comprising instructions for presenting the KPI differences to the user.

16. The non-transitory machine-readable storage medium of claim 7, further comprising instructions for receiving user input to modify the features of similarity and then determining the optimal features of similarity by optimizing the minimum value of an average standard deviation (STD) of the KPI based upon the modified features of similarity.
